(19) United States
(12) Patent Application Publication
(10) Pub. No.: US 2021/0233670 A1
(43) Pub. Date: Jul. 29, 2021

(54) COMPUTERIZED SYSTEM AND METHOD FOR PROVIDING INSTRUCTION AND INCENTIVES TO PHYSICAL THERAPY PATIENTS

(71) Applicant: Intelligent Movement Systems, Truckee, CA (US)

(72) Inventors: Ellen Raynor, San Francisco, CA (US); Mark Feinholz, Redwood City, CA (US); Reed Saunders, Lake Forest Park, WA (US)

(73) Assignee: Intelligent Movement Systems, Truckee, CA (US)

(21) Appl. No.: 17/232,120

(22) Filed: Apr. 15, 2021

Related U.S. Application Data
(63) Continuation of application No. 16/364,172, filed on Mar. 25, 2019.
Application No. 16/364,172 is a continuation-in-part of application No. 15/294,610, filed on Oct. 14, 2016, now abandoned.

(51) Int. Cl. G16H 80/00; G16H 20/30; G06F 3/01; G06F 21/31; G06F 3/0482; G16H 10/60; G06Q 10/10

(52) U.S. Cl. CPC G16H 80/00; G16H 20/30; G06F 3/017; G06Q 10/1097; G06F 3/011; G06F 3/0482; G16H 10/60; G06F 21/31

(57) ABSTRACT

Disclosed is a system and method for transmitting instructions from healthcare providers to patients. In certain embodiments, instructions for completing exercises, prescribed by a physical therapist, are transmitted to patients in the form of video demonstrations with accompanying audio instruction. Other embodiments also incentivize patient compliance with prescribed treatment. Additional embodiments comprise sensors, that detect motion and position, disposed within wearable devices.

### PRIORITY CLAIM

This patent application is a Continuation of U.S. patent application Ser. No. 16/364,172, filed on Mar. 25, 2019, which is a Continuation-in-Part Application of U.S. patent application Ser. No. 15/294,610, filed on Oct. 14, 2016, which claimed the benefit of U.S. Provisional Application 62/242,981 filed on Oct. 16, 2015. These applications are incorporated herein by reference.

### BACKGROUND

There are many instances in which a healthcare provider will provide instructions to patients for activities that patients will perform outside of the direct supervision of the healthcare provider. For example, physical therapists often prescribe exercises for patients that the patients will perform at home. Home exercises have the benefit of speeding recovery time, lowering costs, reducing the likelihood of reinjury, and furthering treatment objectives. One challenge to instructing patients to perform exercises outside of a clinical setting is the possibility a patient will not correctly perform the exercise or forget each step of the exercise. Another challenge faced by providers is keeping patients incentivized to complete their prescribed exercises. Additional challenges are encountered by providers when attempting to accurately document patient activities performed outside of a supervised clinical setting. Disclosed, is a computerized system and method for delivering instructions to patients, providing encouragement to patients, and accurately documenting patient activities.

In certain applications, exercises must be carried out in a specific manner to avoid injury. For example, a person squatting to lift an item should keep his or her hips level and weight distributed such that the knees remain behind the toes. When such exercises are performed without supervision of a clinician, there is a considerable risk that the exercises will not be performed correctly, thereby limiting therapeutic efficacy and increasing the risk of injury. Immediate feedback can prevent injury and increase therapeutic efficacy. Additionally, tracking of movements during exercise can be beneficial for research and training purposes. Certain embodiments of the disclosed invention will detect motion of a user, and identify relative locations and position

### SUMMARY OF THE INVENTION

Certain disclosed embodiments allow a physical therapist, through a computerized system, to select and customize one or more exercises for a patient to perform, and transmit instructions to the patient for each exercise. Certain embodiments display, or provide audio instructions, to the patient an instructional video or audio file of the exercise to be performed as well as a customized do-along video or audio narration for the entire collection of exercises to be performed. Automatic and patient indicated completion of prescribed exercises creates a log as to the date, time, and quality of exercise completion and is then transmitted to the physical therapist. Additionally, certain embodiments will display progress to the patient. In certain embodiments, patient user interfaces are displayed on smartphone or tablet devices.

### FIGURES

FIG. 1 illustrates an embodiment of a user interface through which a provider may select and build a patient profile.

FIG. 2 illustrates an embodiment of a user interface through which a provider is able to view and select a video of an exercise to be transmitted to, and viewed by, a patient.

FIG. 3 illustrates an embodiment of a user interface through which a provider is able to view and customize instructions to be transmitted to, and viewed by, a patient.

FIG. 4 illustrates an embodiment of a user interface through which a provider is able to view and customize details of an exercise for a given patient.

FIG. 5 illustrates an embodiment of an analytic report in which a provider is able to view the patient's activity outside the clinic.

FIG. 6 illustrates an embodiment of a video, viewable on a computer, tablet, smartphone, or an large screen (via devices like AppleTV or ChromeCast) that demonstrates an exercise to a patient.

FIG. 7 illustrates an embodiment of an interface, accessible on a smartphone, wherein a patient is given detailed instructions of their assignment and how to perform an exercise while being tracked by the system.

FIG. 8 illustrates an embodiment of an interface, accessible on a smartphone, wherein a user is able to toggle options over a paused video.

FIG. 9 illustrates an embodiment of an interface, accessible on a smartphone, wherein a patient is able to view a video demonstration of an exercise, and report levels of pain and difficulty to a healthcare provider.

FIG. 10 illustrates an embodiment of an interface, accessible on a smartphone, through which a patient can enter progress into the system by selecting tasks the patient has completed.

FIG. 11 illustrates an embodiment of an interface, accessible on a smartphone, wherein a patient's progress is illustrated with red dots on a calendar to indicate successfully completing an exercise session.

FIG. 12 illustrates an embodiment of an interface, accessible on a smartphone, wherein an overview of a prescribed program of therapy is displayed.

FIG. 13 illustrates an embodiment of an interface, through which a provider can establish, and modify treatment or therapy protocols.

FIG. 14 illustrates an embodiment of an interface, through which a provider can build a patient profile.

FIG. 15 illustrates an embodiment of an interface, through which a provider may create an exercise program that is a circuit program.

FIG. 16 illustrates an embodiment of an interface, through which a provider may add exercises into a circuit within a workout protocol.

FIG. 17 illustrates an embodiment of interface, through which a provider can establish goals and commitments to adhering to a given program.

FIG. 18 illustrates an embodiment of an interface, through which progress and adherence to program goals are displayed to a patient.

### DETAILED DESCRIPTION

Disclosed is a computer system comprising one or more computer networks. Each of the computer networks may be coupled to the Internet. Each network is coupled to one or more computers, accessed by users. Computers, as used herein, includes but is not limited to, laptop devices, tablets, desktop devices, smart phones, smart watches, wearable electronic devices, and all other devices generally understood to have capabilities associated with computers. Users may comprise patients, care providers, relatives of patients, healthcare providers, clinical staff, administrative nursing staff, employers, payers, health systems, or any combination thereof. At least one network is coupled to a server. The server will be coupled to a central repository and one or more computer readable media devices.

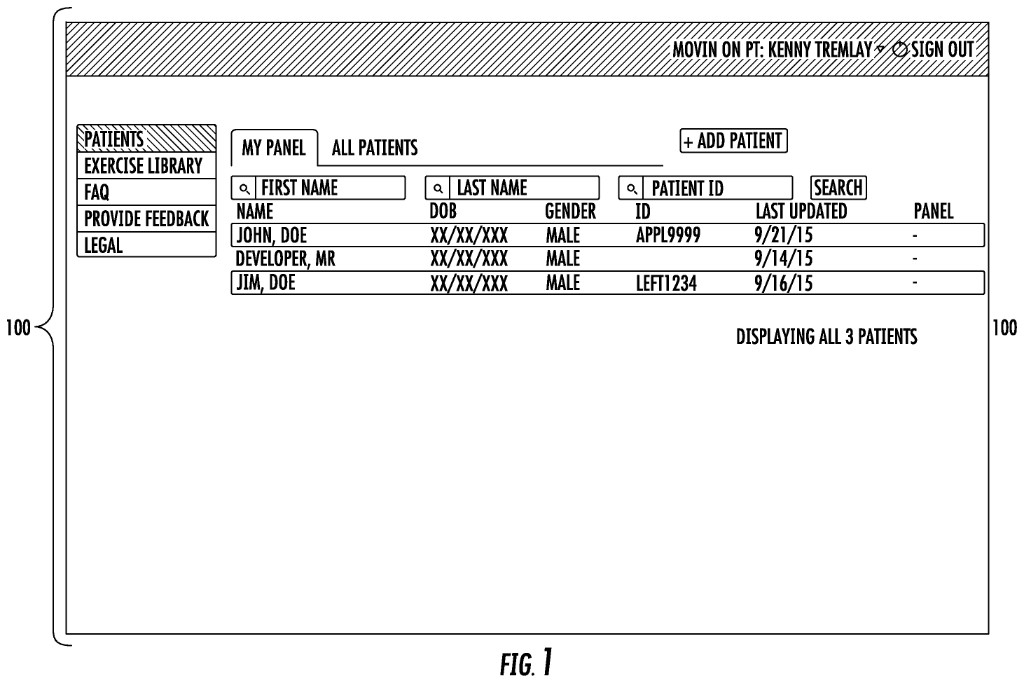

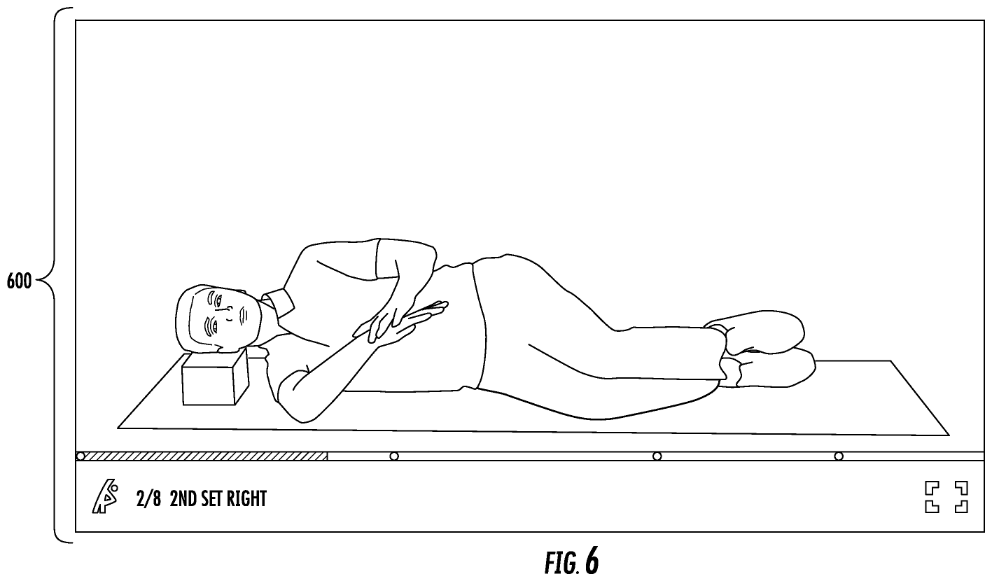

Users access the system through a computer coupled to a network. Users access a user interface by entering a username and password. In alternative embodiments, other credentials may be used to access the system. Once logged onto the system, users are able to access and transmit information stored on the central repository. The user interface may include, but is not limited to, web browser based interfaces, and other software applications, specifically, software applications designed for tablets or smartphones. FIGS. 1-5 and 13-17 illustrate embodiments wherein a user interface is displayed to a healthcare provider and FIGS. 6-12 and 18 illustrate embodiments wherein a user interface is displayed to a patient on a smartphone or tablet device.

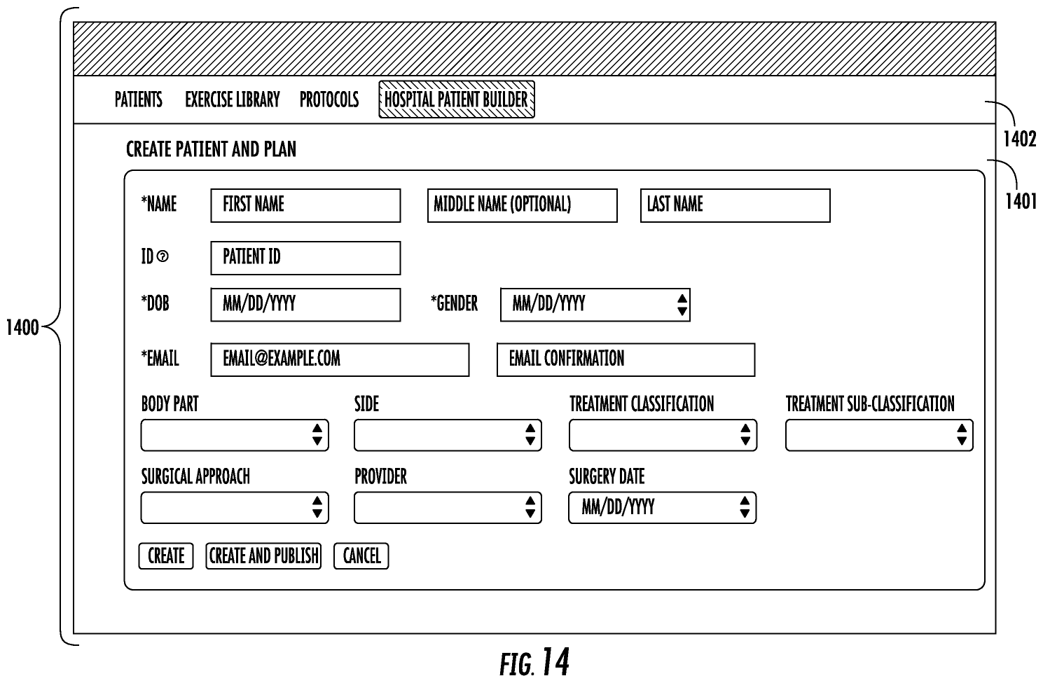

In certain embodiments, different user interfaces exist for healthcare providers and for patients. The user interface provided for healthcare providers allows providers to select patients from an existing panel, or add new patients to the provider's panel, as illustrated in FIG. 1. FIG. 14 illustrates an embodiment wherein a provider may create a patient profile and enter patient information, such as name, applicable ID numbers, date of birth, gender, email address, site of illness or injury, side, treatments, surgical approaches, information regarding other providers, and dates of surgery or other treatments. Providers may create profiles for each patient. Patient profiles include demographic information about each patient, the providers associated with each patient, and health information about each patient. In certain embodiments specific to the physical therapy setting, patient profiles will also list what the rehabilitation program is for each patient, as well as exercises prescribed for that patient.

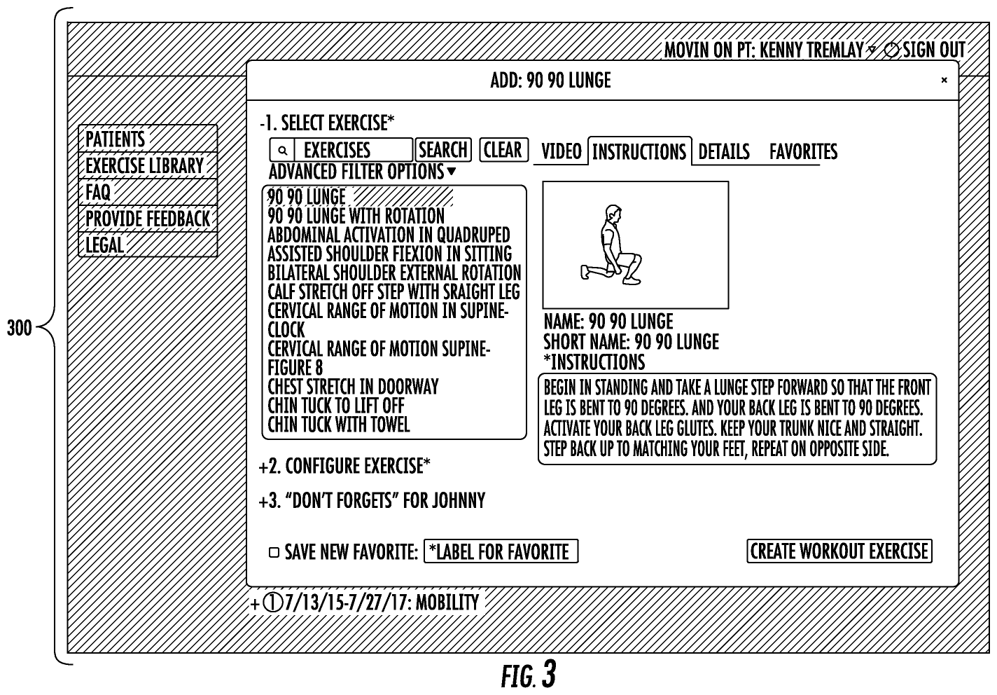

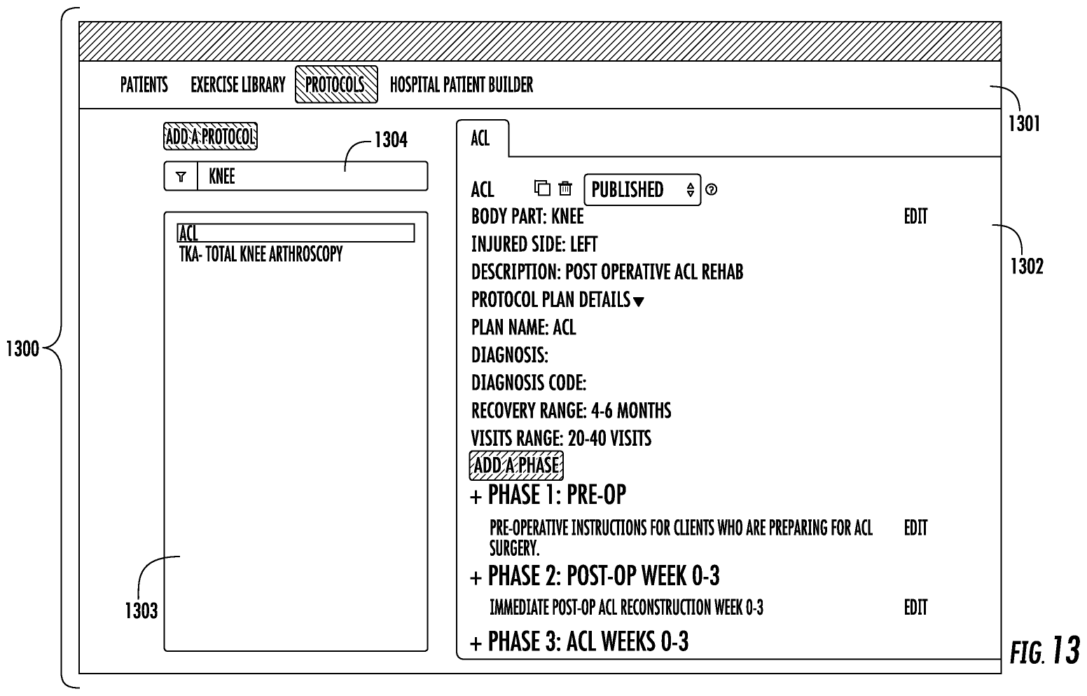

In certain embodiments, a provider can select and transmit instructions to a patient. The examples disclosed herein describe particular utility in physical therapy applications, but the same systems and methods may be utilized in various areas of healthcare and in fields outside of healthcare. In certain embodiments, a provider selects a patient through a user interface. Once the patient is selected, the provider can build an exercise program or a care plan for the patent. In physical therapy applications, a provider will select a given exercise, or a program comprising multiple exercises Once the exercise is selected, the provider will add instructions that are customized for the patient. In certain embodiments, pre-created instructions for common exercises are stored on the central repository, as illustrated in FIG. 3. Providers may select, create and modify, protocols for various treatments. FIG. 13 illustrates an embodiment of a user interface 1300 through which a provider may create a protocol by selecting a given anatomical structure 1304, and a given procedure, injury, or disease state 1303, and a protocol for treatment 1302.

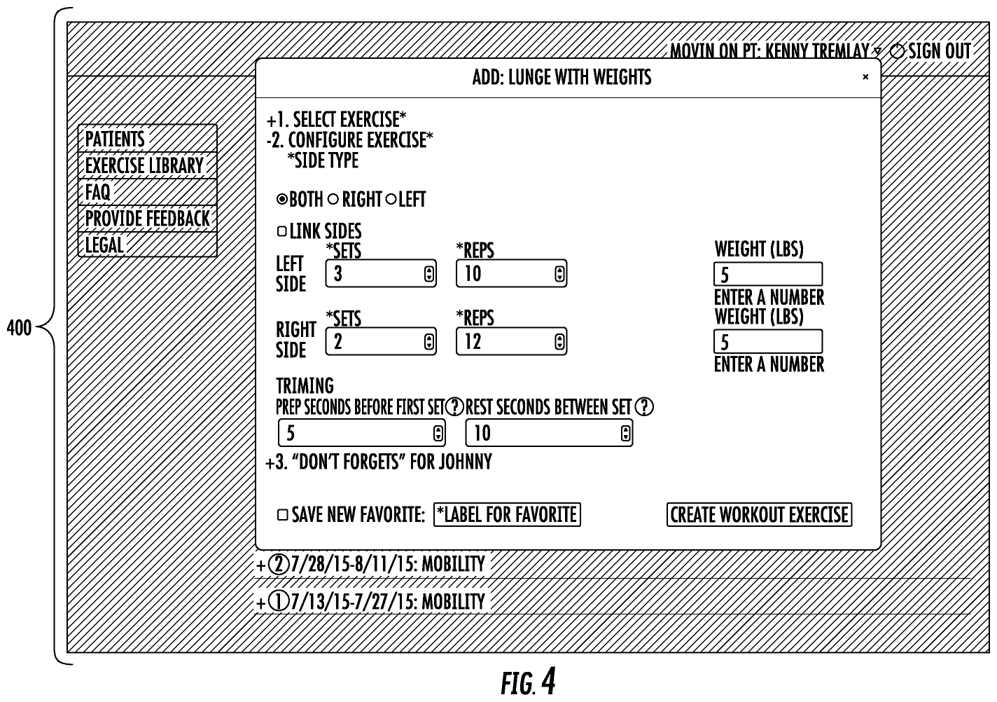

The pre-created instructions may include videos that will demonstrate a given exercise. The pre-created instructions may also comprise one or more template protocols, which may be created by the individual provider. When a provider selects a given exercise for a patient, the provider will complete fields on the user interface to create instructions that are specific for the selected patient as illustrated in FIG. 4. In certain embodiments, providers can specify aspects of a prescribed exercise such as frequency the exercise should be performed, the side that should be exercised (for example, right leg, left arm, etc.), number or repetitions, amount of weight to be used in a given exercise (if applicable), and the duration of time that should be allotted to a given exercise, including any applicable rest periods that should be taken. Exercise instructions typically comprise the following elements: name of the exercise; purpose of the exercise; position of the patient and any related setup required; props or equipment required; where the patient should feel certain sensations; motions that should be completed; motions that are “faulty” or otherwise undesirable; and progression in the exercise program to more advanced exercises or accommodations for a patient's pain or limitations.

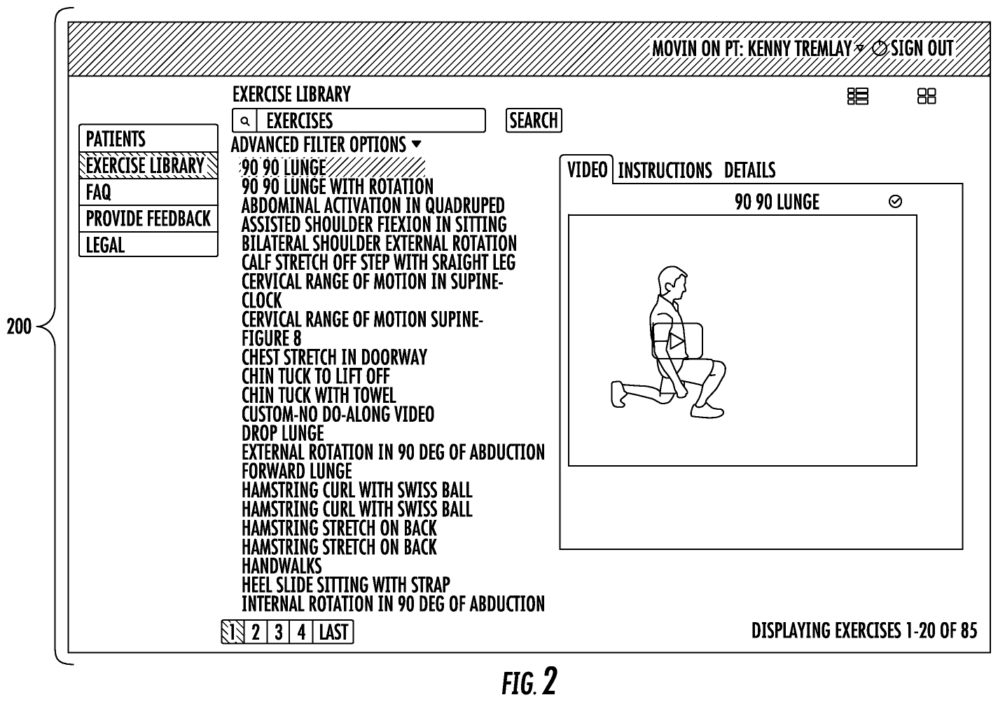

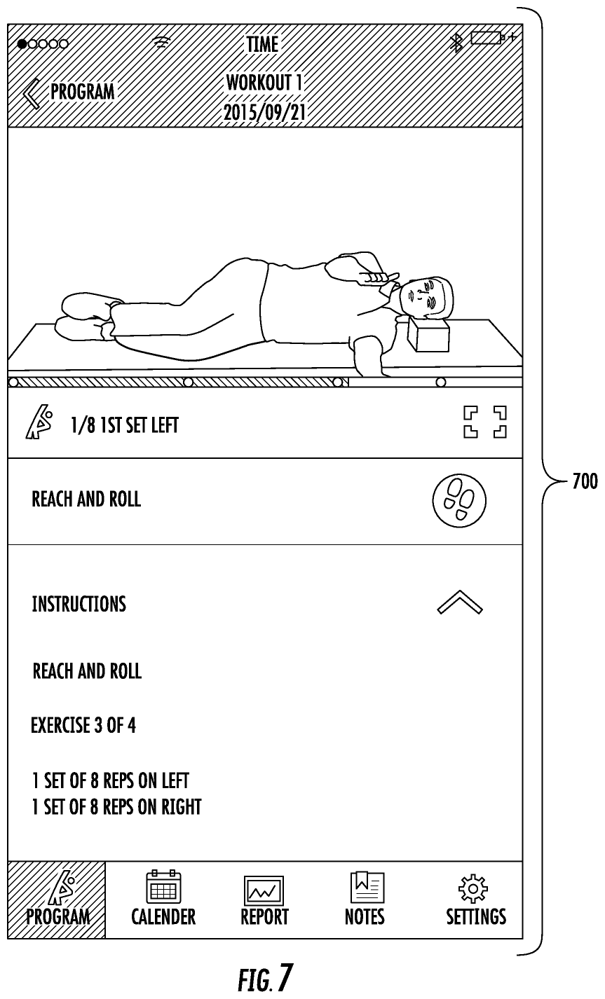

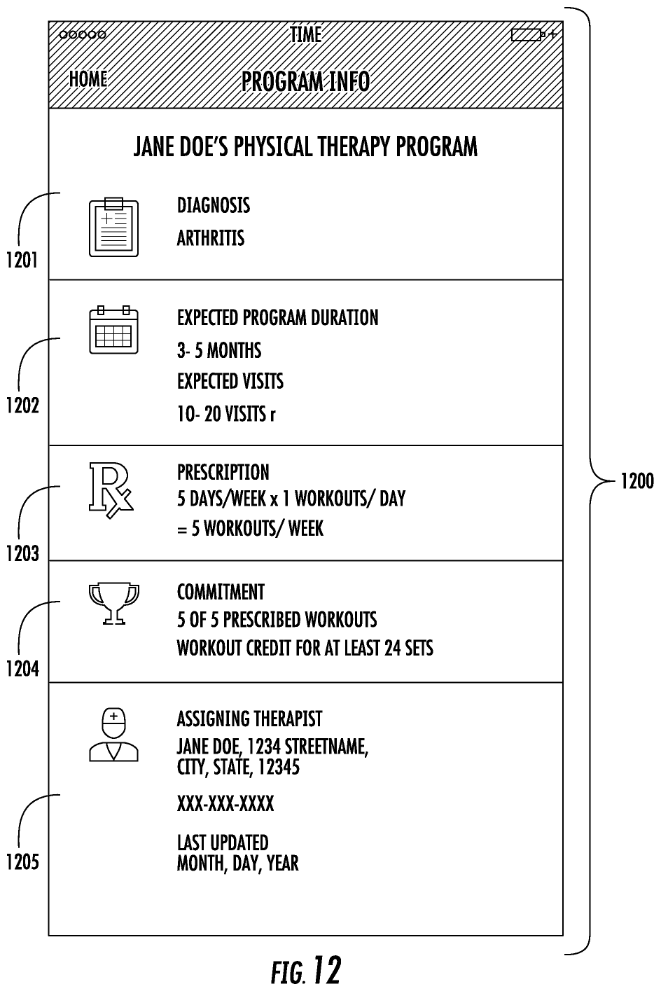

Certain embodiments include videos that patients will see, and audio that patients will hear during their exercise program. FIG. 2 illustrates an embodiment of a user interface wherein a provider may view and select a video for a given patient. FIG. 12 illustrates an embodiment of a user interface wherein details such as patient diagnosis 1201, expected duration of treatment 1202, prescribed exercise frequency 1203, patient commitment 1204, and the provider name and contact information 1205. FIG. 6 illustrates an embodiment of a video, viewable by a patient on a smartphone or tablet device. A key feature of such embodiments is the ability for patients to perform their exercise in a “do-along” format. The videos feature a model performing the prescribed exercise. The videos may be preexisting videos available on the central repository. Providers approve all videos before they are transmitted to a patient for viewing. In such embodiments, when a provider selects an exercise program for a patient, a video accompanying the exercise instructions is prepared for viewing by the patient. The system customizes the video for each patient's individual program. For exercises that have a given number of repetitions, the system will create a loop of the video for the given number of repetitions. In embodiments featuring a video loop, the segment of the video to be repeated is inserted one or more times between an introduction and a conclusion. The number of times the segment is inserted correlates to the number of repetitions to be performed by the patient. Additionally, when exercises require a patient to hold a specified position for a given length of time, the repetitions will be displayed to show a model holding the specified position for the length of time ordered for the patient. This allows the patient to perform exercises in real time with a video featuring a model performing the same exercise with the same number of repetitions. In such embodiments, the patient is reminded exactly how to perform a given exercise while it is being performed as illustrated in FIG. 7. Additionally, during the course of the exercise, the video will keep an accurate count of the repetitions performed by the patients and may automatically track the patient's progress. Videos may also provide patients with audio reminders, some of which may be automatic, and tips during their exercise routine. Finally, in such embodiments, patients may be prompted to log aspects of their experience such as pain or difficulty associated with their prescribed exercise and transmit notes, photos, or videos to their provider. In certain embodiments, other information may be elicited from the patient including the patient's general feeling and the patient's satisfaction or feelings associated with the exercise program.

The system also has auto-mirroring capability wherein the video displayed to a patient will display a model performing the prescribed exercise on the same side as the patient. In such instances, only a single video is uploaded to the central repository, but when a patient has an exercise prescribed for the opposite side demonstrated in the uploaded video, the system will display a mirrored version of the video so it appears that the model is performing the exercise on the same side as the patient. Videos displayed to patients may also keep a count of repetitions and/or time remaining, or that have already been performed, that is displayed during the exercise.

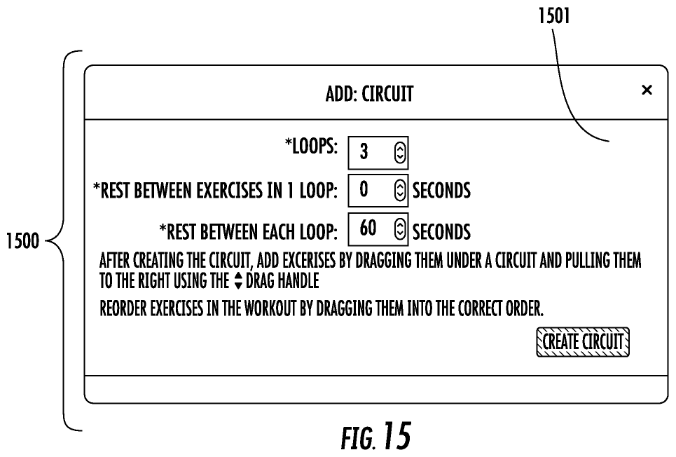

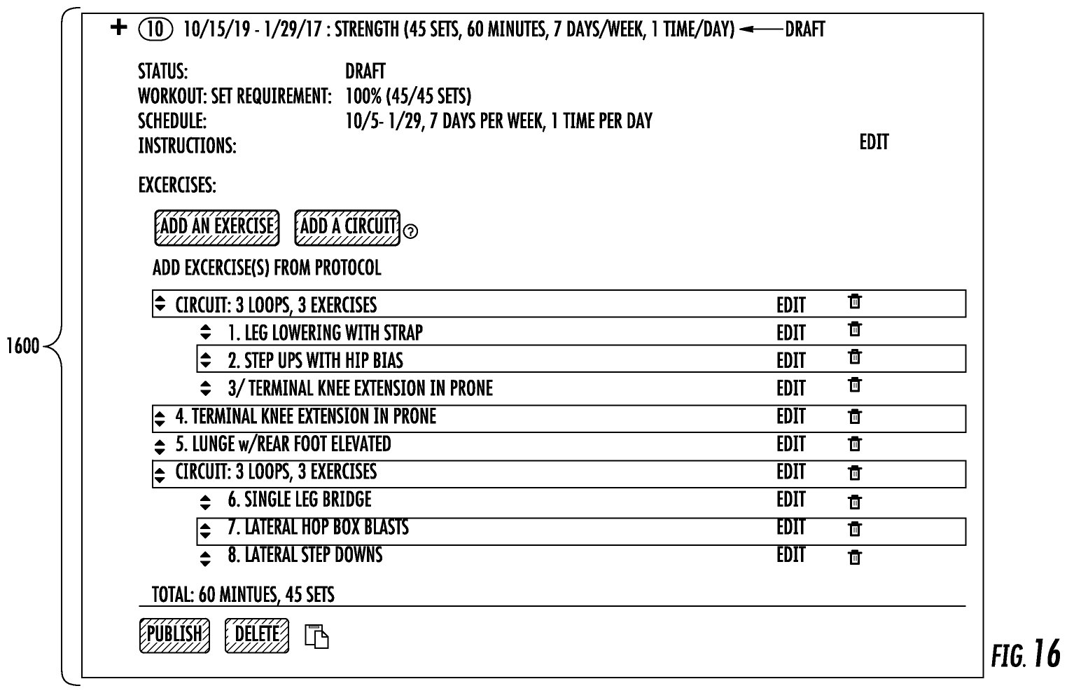

Videos may also feature multiple exercises displayed to patients in orders specified by the provider. In such embodiments, the system will play videos demonstrating multiple specified exercises in an order specified by the provider. Exercises may be ordered serially, or may be ordered in a circuit selected by the provider. FIG. 15 illustrates an embodiment of an interface 1500, through which a provider can create a circuit program for treatment. FIG. 16 illustrates an embodiment of an interface 1600 through which a provider can add exercise or a circuit to a treatment protocol. Certain embodiments may also include exercises that patients can complete independently, without designated times for completion. Patients may also receive reminders for their scheduled exercises, and exercises may be scheduled at varying frequencies.

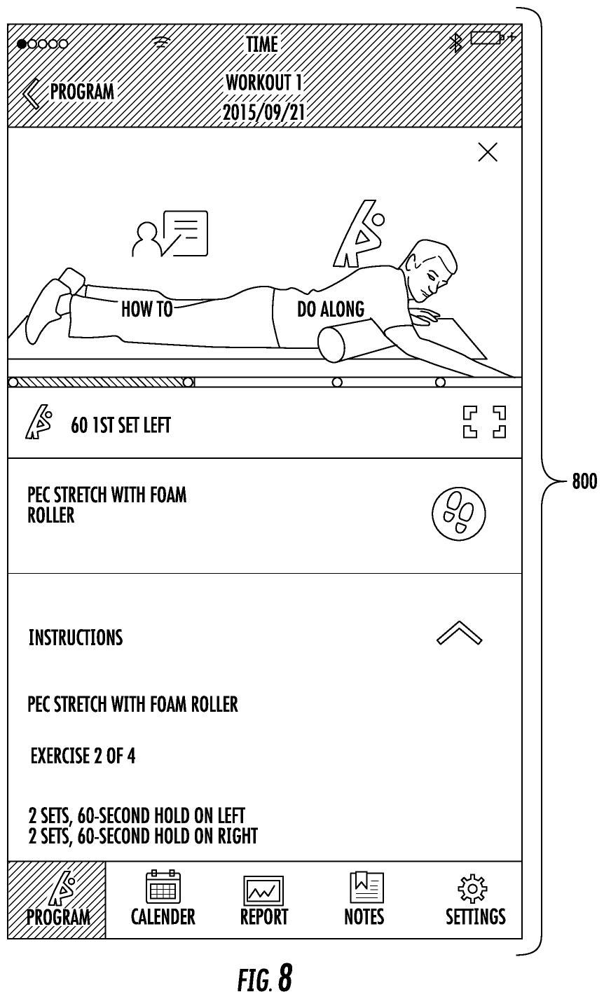

Instructions for performing each exercise are transmitted to the patient along with videos. Patients may read the instructions before beginning the exercise program. While viewing the video, certain embodiments include a toggle function wherein a patient can pause a video and view an instructional video or a “do-along” video, without stopping the first video or having the video start from the beginning. The patient also has the ability to pause or resume the program and the ability to skip exercises. This feature is illustrated in FIG. 8. In certain embodiments, patients who are already familiar with the audio instructions, may listen to music during the workout, while viewing a video demonstrating the correct method of performing a specified exercise.

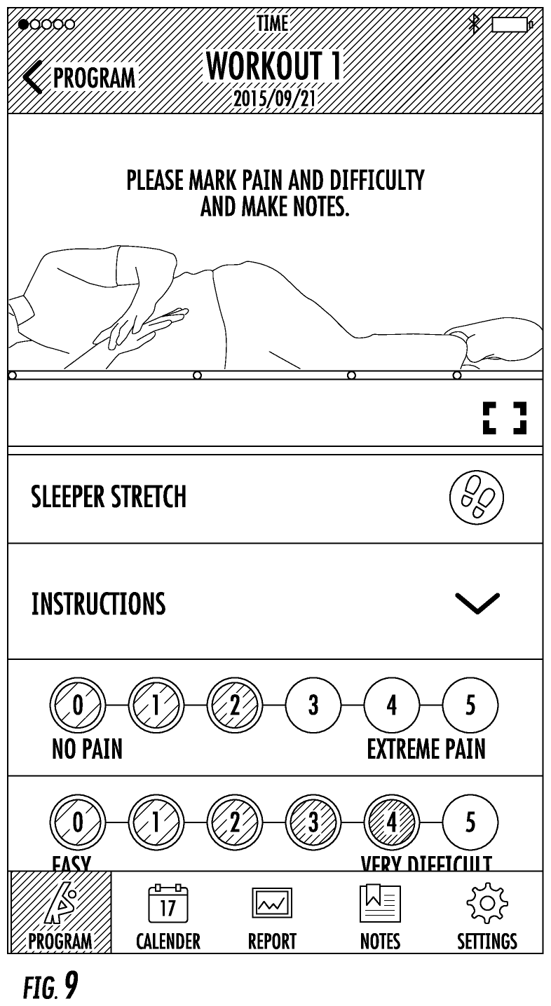

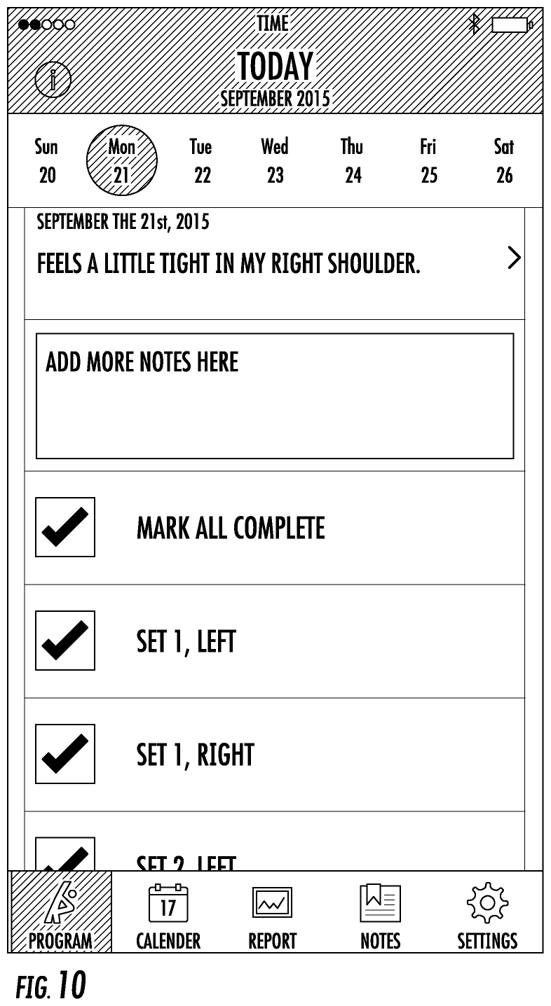

In certain embodiments, patients also have the ability to report pain and difficulty of performing exercises to their provider. FIG. 9 illustrates an embodiment wherein the patient can report pain. In certain embodiments, pain may be reported on a numeric scale. Patients may also report difficulty in completing a given exercise. In certain embodiments, difficulty may be reported on a numeric scale. Patients may also report completion or problems experienced in their program. FIG. 10 illustrates an embodiment wherein a patient is able to report problems and indicate completion or a program.

Providers also have the ability to provide customized instructions to patients. Such instructions may be independent of the video. In certain embodiments, text to speech translation can be performed by the system, thereby creating audio instructions for patients based on text inputs from providers. Providers also have the option of creating their own custom exercise programs. Custom exercise programs may comprise instructions only, instructions and photographs, or may include instructions and custom videos depicting the exercise performed to a given provider's own specifications. In certain embodiments, custom exercise programs may be shared with, and evaluated by, peer healthcare providers.

Patients are able to receive instructions and access to videos through a user interface. Patients access this user interface by providing a username and password, or other credentials. Once logged onto the system, patients are able to view instructions and videos that have been selected by the patient's provider. In certain embodiments, patients will access the user interface with a smartphone or tablet device.

Patients are able to view instructions from their providers and view videos of exercises. Patients are intended to view the videos, and listen to the associated audio files, as they perform the prescribed exercises. Patients also have the ability to log their progress. As illustrated in FIGS. 9 and 10, patients may log each time they perform a prescribed exercise, log the time and day the exercise was performed, indicate whether any pain was experienced during the exercise, and provide other comments or questions to their providers though the user interface. In addition to a log using information provided by the patient, the system may also track patient progress by keeping a record of each video viewed by the patient and the time and frequency each video was viewed by the patient. In such embodiments, the system will keep a record of the patient's progress without the need for the patient to provide any data. The system stores all data entered by patients on the central repository. Data entered by a given patient is viewable to providers who have the given patient in the provider's patient panel. Providers are able to view this data when logged onto the system. Providers may then respond to comments or questions posed by patients. Such responses are stored on the central repository and made available to patients when the patients log onto the system. The system will also track patient activity, such as log in activity, number of times a video has been viewed, dates and times a video was viewed, and whether a video was viewed partially or to completion, and transmit this data to the provider. Patients or providers may provide customized exercise schedules. These schedules will specify times a patient intends to exercise.

Other features may be included in various embodiments. Various enhancements may be provided to the video feature. In certain embodiments, providers may highlight certain attributes of an exercise by using “sportscaster like” custom drawings on a video. In such embodiments, a provider will draw figures directly over a video presentation for viewing by a patient. In other embodiments, providers may provide a different voice track to a video for a given patient, or to all patients in the provider's panel. Providers may also select videos that have specific props, have a specified pace or timing for a given exercise, or display a model in a specific position for a given exercise.

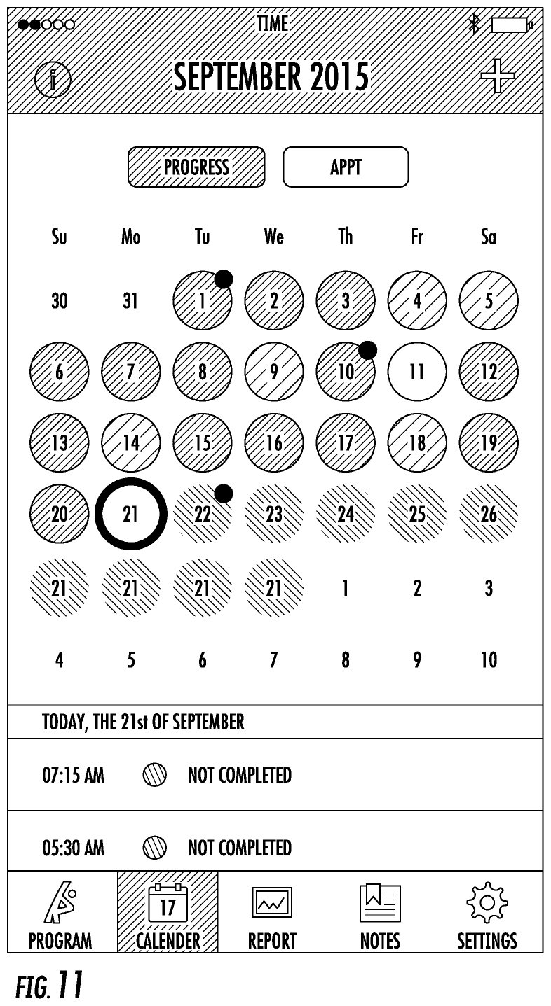

In certain embodiments, the system may also provide encouragement to patients. Encouragement to patients is a key feature in such embodiments. At predetermined points (such as at the end of a given number of repetitions or at the end of an exercise session), encouragement may be provided in the form of an audio comment, music, or a visual graphic displayed to the patient. Additionally, an indicator of “goals-met” may be displayed to patients. In certain embodiments, a red-dot on a calendar will indicate that the patient satisfied certain goals for that day. An example of such an embodiment is illustrated in FIG. 11.

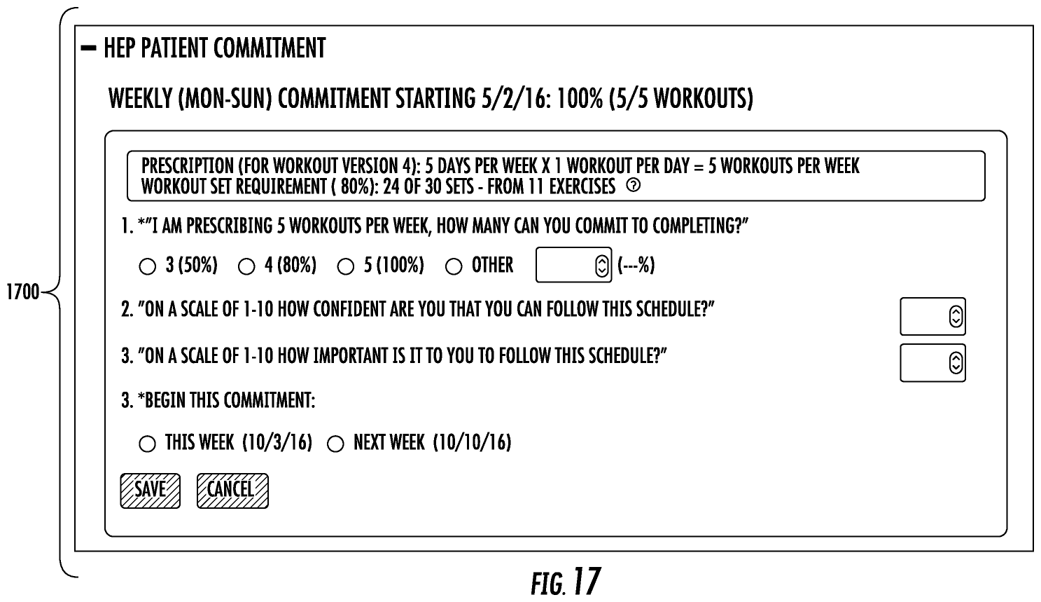

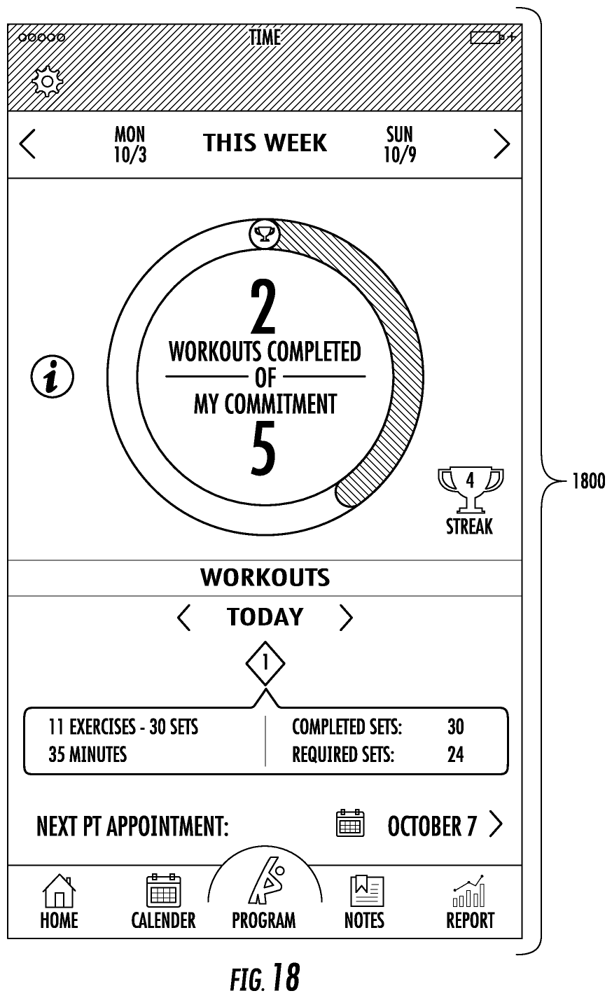

In certain embodiments, the patient and healthcare provider may engage in collaborative goal setting through motivational interviewing techniques, and those goals may involve heterogeneous objectives. The healthcare provider will specify the frequency of instructions such as twice a day seven days a week, but then set a target commitment that the patient agrees is important and achievable. For example, a provider and patient may agree on a target, such as a percentage that translates into 10 workouts per week. FIG. 17 illustrates an embodiment of an interface 1700, through which a patient can indicate agreement and commitment to adherence to a prescribed program. Furthermore, workouts may be heterogeneous tasks. Unlike step counts or mileage, there may be multiple different workouts in a week, and workouts contain a collection of activities organized into sets. In certain embodiments, credit for a workout can be customized to a percentage of sets completed. In many instances, it would not be reasonable to get credit for a workout where only 5% has been completed. Similarly, it would be reasonable to get credit for a workout where 90% of the sets were completed. The instructions and goals may change over time without impacting the achievement of previous goals or streaks of weeks in which the goals have been met. FIG. 18 illustrates an embodiment of a user interface 1800 wherein a patient's progress and adherence are displayed.

In certain embodiments, patients may also customize videos by selecting music that can be played during a video. Music may be selected from the system, from a local device, or from an external service. Patients may also pause videos at certain points to pause their exercise routine, and resume the video when they wish to resume their exercise at the same point in the video. Certain embodiments also allow a patient to access videos and instructions when not connected to the internet. In such embodiments, patients will download one or more videos and instructions for one or more exercises and store such the videos and instructions on a local device, which may be a smartphone or tablet device.

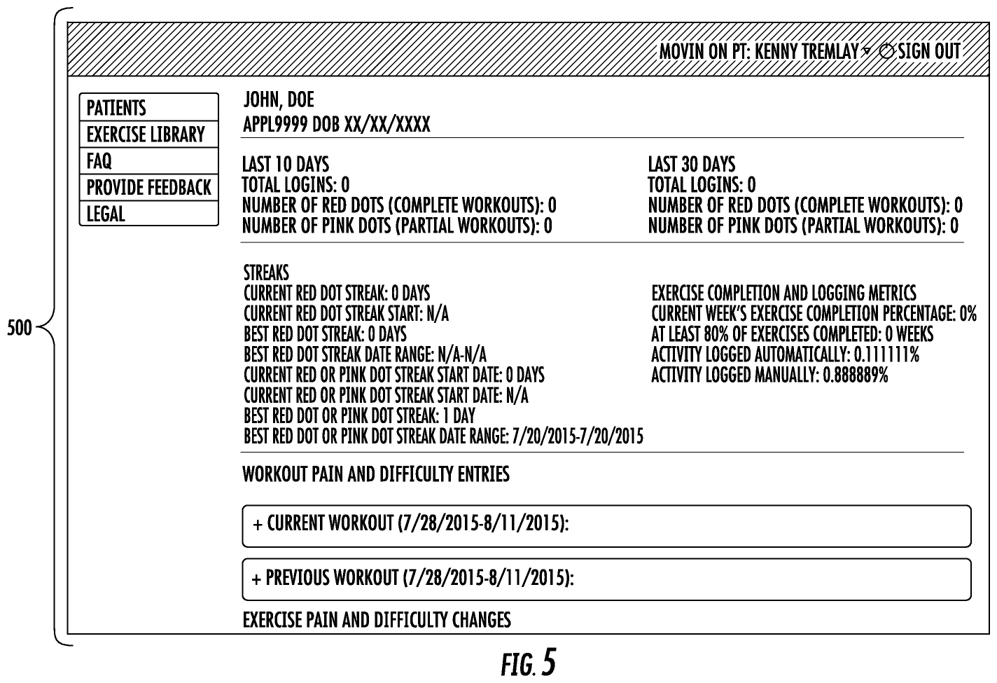

Certain embodiments will also interface directly with an Electronic Health Record (EHR). In such embodiments, the system will obtain patient information from the EHR and prepopulate the fields that a provider would normally enter. Exercises prescribed for each patient will also be uploaded directly to the EHR. In certain embodiments, patient data, such as the exercises the patient has completed, time and date of completion, and any patient comments will also be uploaded directly to the EHR. FIG. 5 illustrates an embodiment of how information about a patient experience outside the clinic might be viewed by the healthcare provider. In certain embodiments, de-identified patient progress data may be aggregated across diagnosis codes, workout programs, and healthcare providers to discover trends.

Certain embodiments will allow patient management by a care team. It is common for a patient to be treated by multiple healthcare providers as part of an episode of care. It is also common for a patient to switch providers. In such embodiments, the patient may invite a provider into the system to access and modify patient instructions while maintaining a clear record of which provider made which modifications to the instructions. Additionally, the patient may specify which existing providers will have access to instructions from new providers. The patient may also invite caregivers such as family members to view instructions as the patient's proxy.

In certain embodiments, the system will receive input from various input devices. Such devices include, but are not limited to, wearable devices (such as FitBit, wearable heart rate monitors, etc.) that are able to obtain data (such as vital signs, distance traveled, temperature, etc.). Users may use multiple wearable devices having motion and location sensors. The wearable devices may include sensors to detect motion and location relative to other sensors. The wearable devices including sensors may be disposed on a user's body with a hook and loop system, magnets, springs, elastic bands, adhesive, belts, straps, bracelets, necklaces, or any other means of securing a device to one or more locations on a user's body. Additionally, such wearable devices may also include hardware to determine if the device is level relative to the ground or another sensor. In certain embodiments, wearable devices may also determine the location and motion of a user or a user's body part. For example, a wearable device on a user's wrist or arm may determine the range of motion at a user's shoulder, elbow, wrist, or other upper extremity structure. Additionally, a user may have multiple sensors in wearable devices In such embodiments, data is automatically submitted to the system.

When user data is obtained through wearable devices, information is transmitted wirelessly to the system. The wireless devices having sensors are coupled to the system wireless through Bluetooth or WiFi connections. Data accumulated from the wearable devices can be stored on the system and transmitted to healthcare providers to monitor a user's progress, to provide feedback, or to assess or diagnose a user. Additionally, the system can be configured to provide instantaneous feedback to a user to correct incorrect movements during an exercise. Either a healthcare provider or a user may configure system settings to alert a user to motions that may be out of a specified range or when a given motion exceeds, or fails to meet, a predetermined threshold. Such ranges and thresholds may include the extent to which a user or body part is level or whether exercises are being performed symmetrically. Such settings are set through a user interface. In such embodiments, a visual alert may be displayed on a user interface, an audio alert may be generated, or a device may vibrate.

While the invention has been described and illustrated with reference to particular embodiments thereof, those skilled in the art will appreciate that the various adaptations, changes, modifications, substitutions, deletions, or additions or procedures and protocols may be made without departing from the spirit and scope of the invention. Specifically, while particular utility has been disclosed in healthcare, skilled artisans will appreciate that the systems and methods disclosed herein may be utilized across many fields of use. It is intended, therefore, that the invention be defined by the scope of the claims that follow and that such claims be interpreted as broadly as reasonable.

What is claimed is:

1. A method for transmitting information to and from patients comprising:
obtaining a username and password from a provider, through a computer network;
receiving a selection of a patient from the provider through a computer network;
displaying a plurality of exercises to a provider, each exercise having instructions and a video associated with said exercise;
obtaining a username and password from a patient though a computer network;
displaying the instructions and the video to the patient, said instructions and video being associated with the exercise selected by the provider;
logging whether the patient viewed the instructions and video on the central repository;
obtaining data from sensors disposed within wearable devices on the patient; and
displaying a log of patient activity to a provider, said log comprising dates and times a patient viewed the instructions or the video.

2. The method of claim 1 further comprising the steps of:
obtaining from a provider a specified time and frequency for the exercise;
displaying the specified time and frequency to the patient; and
displaying to the patient a goals-met indicator on a user interface.

3. The method of claim 1 further comprising the steps of:
receiving a number of repetitions for the exercise from the provider; and
displaying a video having the number of repetitions received from the provider to the patient.

4. The method of claim 3 further comprising the steps of:
receiving a specified side for an exercise from the provider; and
displaying a video demonstrating an exercise on the side specified by the provider to the patient.
